(19) United States
(12) Patent Application Publication
(10) Pub. No.: US 2024/0382098 A1
(43) Pub. Date: Nov. 21, 2024

(54) DEVICE AND METHOD FOR MEASURING BLOOD PRESSURE AND COMPUTER PROGRAM PRODUCT THEREOF

(71) Applicant: NexVita Technology Corp., Taoyuan City (TW)

(72) Inventors: Kun-I LIN, Taoyuan City (TW); Pei Yuan Tsai, Taoyuan City (TW); Shuo Cheng Chou, Taoyuan City (TW)

(21) Appl. No.: 18/369,882

(22) Filed: Sep. 19, 2023

(30) Foreign Application Priority Data
May 19, 2023 (TW) 112118726

(51) Int. Cl. A61B 5/021; A61B 5/00

(52) U.S. Cl. CPC A61B 5/02108; A61B 5/7203; A61B 5/725

(57) ABSTRACT

A device for measuring blood pressure receives a vibration signal from a vibration sensor which is used to measure a target area. The device then converts the vibration signal into a digital signal and performs a filtering process on the digital signal. The filtering process involves removing noise around the principal component wave that corresponds to the pulsation of the target area within a specific range in the digital signal. Subsequently, based on the digital signal that has been filtered, the device determines a systolic pressure determination time point and a diastolic pressure determination time point to generate a blood pressure measurement result.

### CROSS-REFERENCE TO RELATED APPLICATIONS

This application claims priority to Taiwan Patent Application No. 112118726 filed on May 19, 2023, which is hereby incorporated by reference in its entirety.

### BACKGROUND OF THE INVENTION

The present disclosure relates to a blood pressure measurement technology. More specifically, the present disclosure relates to a device and a method for non-invasive blood pressure measurement and a computer program product thereof.

When bounding and pressurizing a specific blood vessel position (e.g., the brachial artery) in the human body and then release the pressure, the blood therein that was stopped from flowing at first will flow through the blood vessel position again, during which the blood rubs and collides with the blood vessel wall and causes vibration of the blood vessel wall. A sound referred to as “Korotkoff sound” can be observed when the vibration of the blood vessel wall is transmitted to the body surface. This concept was first introduced by the Russian physician Nikolai Sergeievich Korotkoff in 1905 and thus is named after him. Korotkoff sound can be roughly divided into five stages from its generation to its disappearance, each with different sound characteristics that represent different vascular and/or blood flow conditions. By observing the pressure during depressurization and the appearance of Korotkoff sound in each stage, a measurer (i.e., a person for measuring blood pressure) can evaluate the systolic pressure (which is said to correspond to the pressure measured when Korotkoff sound first appears) and the diastolic pressure (which is said to correspond to the pressure measured when Korotkoff sound finally disappears) accordingly. Therefore, Korotkoff sound is an identifiable reference in the practice of non-invasive blood pressure measurement.

Most of the traditional non-invasive blood pressure measurement techniques rely on the measurers to listen to the epidermal position corresponding to a specific blood vessel position through sound reception means such as a stethoscope or a microphone, and distinguish the stages of Korotkoff sound from the sound based on their experience, thereby determining the time points for determining the systolic pressure and the diastolic pressure. However, such a way of identification is merely based on experience with no objective standards, and the listening process thereof is very susceptible to external noise interference. In view of this problem and considering that Korotkoff sound originates from the vibration of the blood vessel wall, theoretically, waveforms consistent with the state of Korotkoff sound can be obtained through measuring the vibration of the body surface, so as to avoid the interference of external noise in the measurement process.

For example, the technology of measuring blood pressure through a pressure sensor was mentioned in U.S. patent application Ser. No. 11/370,020. The U.S. patent application Ser. No. 11/370,020 adopts vibration-related means to generate a waveform diagram, and determines the systolic pressure determination time point and the diastolic pressure determination time point according to the appearance and disappearance of a specific type of notch in the waveform shown in the waveform diagram (e.g., a notch N1 in the waveform W1 shown in FIG. 5, details of which will be described later.) However, there is much noise (e.g., notches N2 and N3 in the waveform W1 shown in FIG. 5, details of which will be described later) in the waveform generated by the technology described in, for example, the U.S. patent application Ser. No. 11/370,020. Whether the notches are discerned through human observation or machine vision, the noise is highly likely to cause misjudgment, which further impacts the precision of subsequent cardiovascular treatments, such as blood pressure measurement and arteriosclerosis index calculation, or the like.

Accordingly, there is an urgent need in the art for a new way to provide a method for blood pressure measurement which effectively suppresses the above-mentioned noise.

### SUMMARY OF THE INVENTION

To solve at least the above-mentioned problem, the present disclosure provides a device for measuring blood pressure. The device for measuring blood pressure may comprise a signal conversion circuit and a processor electrically connected with the signal conversion circuit. The signal conversion circuit may be configured to receive a vibration signal from a vibration sensor, and the vibration signal may be generated by the vibration sensor by measuring a target area. Moreover, the signal conversion circuit may also be configured to convert the vibration signal into a digital signal. The processor may be configured to perform a filtering process on the digital signal. The filtering process comprises filtering out the noise around a principal component wave corresponding to the pulsation of the target area within a specific range in the digital signal. Moreover, the processor may also be configured to determine a systolic pressure determination time point and a diastolic pressure determination time point according to the digital signal that has been filtered so as to generate a blood pressure measurement result.

Furthermore, the filtering process may further filter out signal components with frequencies higher than 70 Hz in the digital signal.

Furthermore, the filtering process may further filter out signal components with frequencies lower than 15 Hz in the digital signal.

Furthermore, the signal conversion circuit may comprise three resistors, the three resistors constitute a Wheatstone bridge with the vibration sensor, and the signal conversion circuit further comprises a differential signal amplifier electrically connected with the Wheatstone bridge, a low-pass filter electrically connected with the differential signal amplifier, and an analog-to-digital converter electrically connected with the low-pass filter and the processor. The Wheatstone bridge may convert the vibration signal into a pair of differential signals, while the differential signal amplifier may be configured to receive the pair of differential signals from the Wheatstone bridge and convert the pair of differential signals into an amplified signal. The low-pass filter may be configured to filter the amplified signal. The analog-to-digital converter may be configured to convert the amplified signal that has been filtered into the digital signal.

Furthermore, the device for measuring blood pressure may further comprise a pulse pressing element and an air pressure sensing circuit electrically connected with the processor and the pulse pressing element. The processor may be further configured to control the pulse pressing element to exert pressure on the target area, and the air pressure sensing circuit may be configured to generate a pressure signal corresponding to the pulse pressing element and provide the pressure signal to the processor. Moreover, the processor generates the blood pressure measurement result according to the systolic pressure determination time point, the diastolic pressure determination time point and the pressure signal.

Furthermore, the processor may perform the filtering process by implementing a finite impulse response digital filter.

Furthermore, the device for measuring blood pressure may further comprise the vibration sensor, and the vibration sensor may comprise a metallic diaphragm. Moreover, the vibration sensor may be a piezoresistive strain gauge, and the metallic diaphragm may be made of copper alloy. Further speaking, the material of the metallic diaphragm may be phosphor bronze.

To solve at least the above-mentioned problem, the present disclosure further provides a method for measuring blood pressure. The method may be executed by a device for measuring blood pressure, and may comprise the following steps:
receiving a vibration signal from a vibration sensor, wherein the vibration signal is generated by the vibration sensor by measuring a target area;
converting the vibration signal into a digital signal;
performing a filtering process on the digital signal, wherein the filtering process comprises filtering out the noise around a principal component wave corresponding to the pulsation of the target area within a specific range in the digital signal; and
determining a systolic pressure determination time point and a diastolic pressure determination time point according to the digital signal that has been filtered so as to generate a blood pressure measurement result.

To solve at least the above-mentioned problem, the present disclosure further provides a computer program product. The computer program product, after being loaded into an electronic computing device, may cause the electronic computing device to execute the method for measuring blood pressure described above according to the present disclosure.

According to the above description, the device and method for measuring blood pressure and the computer program product thereof provided according to the present disclosure can eliminate the noise surrounding the principal component wave corresponding to the pulsation within the digital signal derived from the vibration sensor. This filtration process results in a signal of which the characteristics tend to be consistent with the Korotkoff sound outcomes, and thus the noise level is lower than that in the prior art. Therefore, the device and method for measuring blood pressure and the computer program product thereof provided according to the present disclosure indeed improve the long-standing technical problem that noise affects blood pressure judgment in the technical field (as mentioned earlier), and meanwhile, they can further improve the accuracy of subsequent cardiovascular condition evaluation.

This summary section describes the overall concept of the present disclosure, and covers problems that can be solved by the present disclosure, means that can be adopted by the present disclosure and effects that can be achieved by the present disclosure, so as to provide a basic understanding of the present disclosure for those of ordinary skill in the art. However, it shall be appreciated that, these paragraphs in the summary section are not intended to summarize all embodiments of the present disclosure, but only present the core concept of the present disclosure in a simple form as an introduction to the detailed description that follows. Hereinafter, the detailed technology and implementation of the present disclosure will be described with reference to attached drawings, so that a person having ordinary skill in the art can understand the technical features claimed in the present invention.

### BRIEF DESCRIPTION OF DRAWINGS

As shown below:

FIG. 1 is a schematic view depicting a device for measuring blood pressure according to one or more embodiments of the present disclosure.

FIG. 2 is a schematic view depicting a vibration sensor in the device for measuring blood pressure shown in FIG. 1.

FIG. 3 is a schematic view depicting a signal conversion circuit in the device for measuring blood pressure shown in FIG. 1.

FIG. 4 is a schematic view depicting an air pressure sensing circuit in the device for measuring blood pressure shown in FIG. 1.

FIG. 5 is a schematic view depicting waveforms generated by a device for measuring blood pressure according to one or more embodiments of the present disclosure and waveforms generated by the prior art.

FIG. 6 is a flowchart diagram depicting a method for measuring blood pressure according to one or more embodiments of the present disclosure.

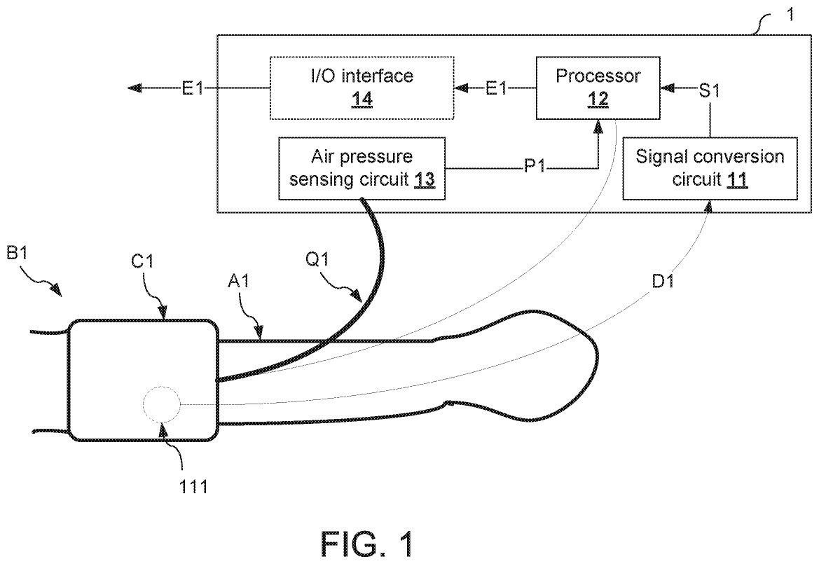

The contents shown in FIG. 1 to FIG. 6 are only exemplary examples for explaining the embodiments of the present disclosure and are not intended to limit the scope claimed in the present disclosure.

### DETAILED DESCRIPTION OF THE INVENTION

Hereinafter, the device and method for measuring blood pressure and the computer program product thereof provided according to the present disclosure will be explained through embodiments. However, these embodiments are not intended to limit the present disclosure to any environments, applications or implementations described in these embodiments. Therefore, description of these embodiments is only for purpose of explaining the present disclosure rather than for limiting the scope of the present disclosure. It shall be appreciated that, in the following embodiments and the attached drawings, elements unrelated to the present disclosure are omitted from depiction; and dimensions of and dimensional scales among individual elements are provided only for illustration, but not to limit the scope of the present disclosure.

Please refer to FIG. 1. A first embodiment of the present disclosure is a device 1 for measuring blood pressure, which may at least comprise a signal conversion circuit 11 and a processor 12 electrically connected to the signal conversion circuit 11. In general, the signal conversion circuit 11 may be configured to receive a vibration signal D1 from a vibration sensor 111 and convert the vibration signal D1 into a digital signal S1. The processor 12 may be configured to perform a filtering process on the digital signal S1 and determine a systolic pressure determination time point and a diastolic pressure determination time point according to the digital signal S1 that has been filtered, so as to generate a blood pressure measurement result E1.

The processor 12 may be one of microprocessors or microcontrollers or the like capable of processing signals. The microprocessors or microcontrollers are a kind of programmable special integrated circuits which are capable of computing, storing, outputting/inputting or the like, and they can accept and process all kinds of coded instructions to perform various logical operations and arithmetic operations and output corresponding operation results. The processor 12 may be programmed to interpret various instructions to process data in the device 1 for measuring blood pressure and execute various arithmetic procedures or programs.

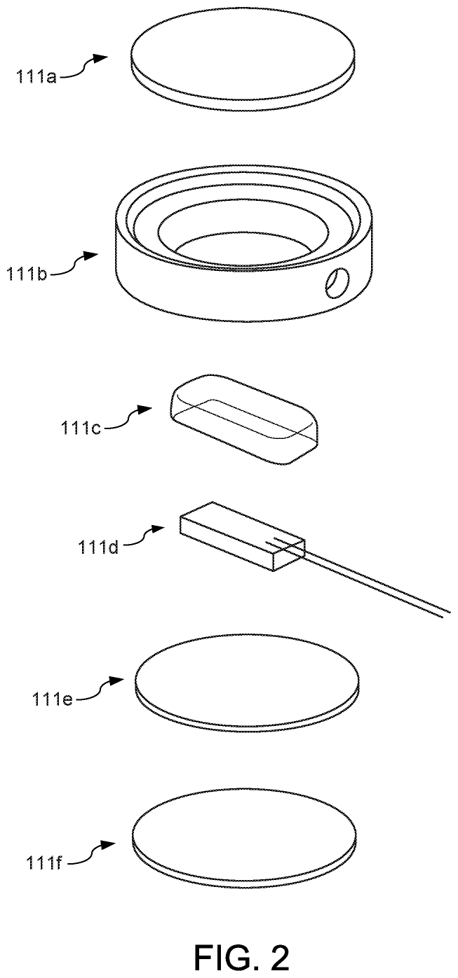

Please refer to FIG. 2, which depicts an example of the structure of the vibration sensor 111. In some embodiments, the vibration sensor 111 may at least comprise a strain gauge 111d and a metallic diaphragm 111e. The metallic diaphragm 111e may be attached to the strain gauge 111d.

In some embodiments, the thickness of the metallic diaphragm 111e may be in the range of 0.1 to 0.2 cm. In some embodiments, the metallic diaphragm 111e may be made of copper or copper alloy. In some embodiments, the copper alloy may be phosphor bronze. Furthermore, in some embodiments, the strain gauge 111d may be a piezoresistive strain gauge.

In order to protect the strain gauge 111d and the metallic diaphragm 111e, the vibration sensor 111 may further comprise a protective cover 111c covering the strain gauge 111d, a housing 111b surrounding the strain gauge 111d and the metallic diaphragm 111e, and a back cover 111a covering the housing. In addition, in some embodiments, the vibration sensor 111 may further comprise a silicone diaphragm 111f placed under the metallic diaphragm 111e to avoid direct contact between the metallic diaphragm 111e and the target area.

Referring to FIG. 1 and FIG. 2 together, the vibration sensor 111 may be arranged to be closely attached to a target area of a subject, such as an arm A1 shown in FIG. 1, and may generate a corresponding vibration signal D1 by sensing the vibration of the target area (e.g., at the position of the brachial artery).

In some embodiments, thanks to the way of sensing of the strain gauge 111d, the device 1 for measuring blood pressure can avoid being affected by the environmental noise during the measurement. In addition, in some embodiments, thanks to the material selection of the metallic diaphragm 111e, the device 1 for measuring blood pressure has more lenient requirements for the positioning and movement of the subject. For example, as tested by the inventor, the metallic diaphragm 111e made of phosphor bronze allows the subject to moderately engage in normally activities (e.g., move his/her arm) in the process of blood pressure measurement without the need for complete stillness.

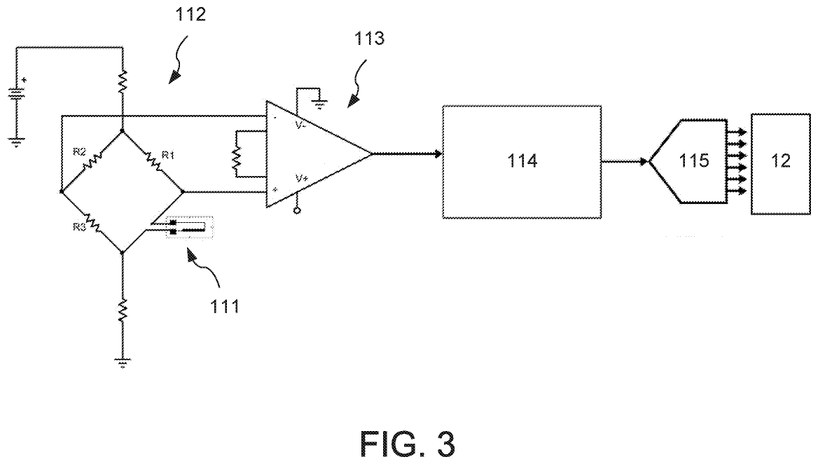

The vibration signal D1 generated by the vibration sensor 111 may then be provided to the signal conversion circuit 11 to be converted into a digital signal. Referring to FIG. 3, the signal conversion circuit 11 may comprise three resistors R1, R2, and R3. These three resistors R1, R2, and R3 may constitute a Wheatstone bridge 112 with the vibration sensor 111. The Wheatstone bridge 112 may convert the vibration signal D1 into a set of differential signals, and the signal conversion circuit 11 may further comprise a differential signal amplifier 113 electrically connected with the Wheatstone bridge 112, and the differential signal amplifier 113 may be configured to receive the pair of differential signals from the Wheatstone bridge 112 and convert the pair of differential signals into an amplified signal. The signal conversion circuit 11 may further comprise a low-pass filter 114 electrically connected to the differential signal amplifier 113 and it may be configured to filter the amplified signal.

In addition, the signal conversion circuit 11 may further comprise an analog-to-digital converter 115 electrically connected with the low-pass filter 114 and the processor 12, and the analog-to-digital converter 115 may be configured to convert the amplified signal that has been filtered into the digital signal S1 and provide the digital signal S1 to the processor 12. In some embodiments, the digital signal S1 is composed of a series of discrete values relative to the amplified signal obtained by the analog-to-digital converter 115 through continuously sampling the amplified signal according to a sampling frequency.

The details and implementation of the low-pass filter 114 and the analog-to-digital converter 115 in FIG. 3 shall be appreciated by those of ordinary skill in the art, and thus will not be further described in the present disclosure.

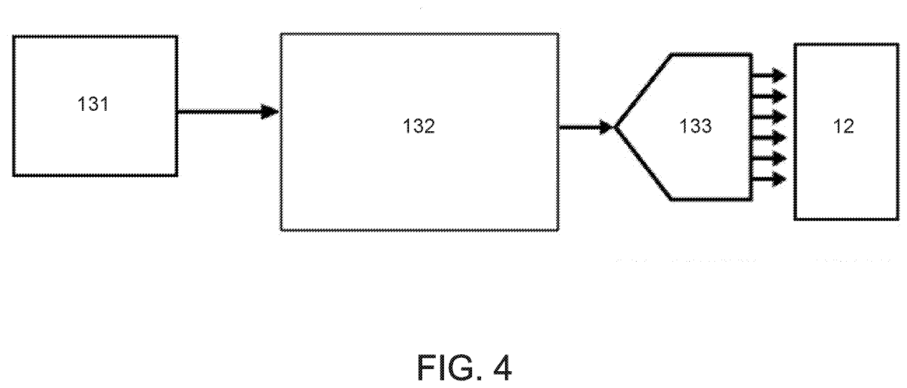

Please refer to FIG. 1 and FIG. 4 together. In some embodiments, the device 1 for measuring blood pressure may further comprise a pulse pressing element B1 and an air pressure sensing circuit 13 electrically connected with the processor 12 and the pulse pressing element B1. The pulse pressing element B1 may comprise a pulse pressing belt C1, an air pipe Q1, an inflator pump (not shown in the figure), and a deflation valve (not shown in the figure). The air pressure sensing circuit 13 may comprise an air pressure sensor 131, a low-pass filter 132 coupled with the air pressure sensor 131, and an analog-to-digital converter 133 coupled with the low-pass filter 132. The details and implementation of the air pressure sensor 131, the low-pass filter 132 and the analog-to-digital converter 133 in FIG. 4 shall be appreciated by those of ordinary skill in the art, and thus will not be further described in the present disclosure.

The pulse pressing belt C1 may have a space in which the vibration sensor 111 may be accommodated, so that the vibration sensor 111 may be disposed in the space. Alternatively, the vibration sensor 111 may be fixed to the pulse pressing belt C1 by adhesive attachment, fastening, magnetic attraction or the like.

The inflator pump may have a structure in which a piston is driven by an electric motor, and it can inflate the pulse pressing belt C1 through the air pipe Q1. The deflation valve may be an air valve of an electromagnetic structure, which can stably deflate the pulse pressing belt C1 after inflation.

The processor 12 may be configured to control the inflator pump to inflate the pulse pressing belt C1 through the air pipe Q1, thereby applying pressure to the target area to block the blood circulation of blood vessels, and then control the deflation valve to release the air in the pulse pressing belt C1 stably, thereby gradually reducing the pressure applied to the target area. In the process of pressurization and depressurization, the air pressure sensor 131 in the air pressure sensing circuit 13 may generate a corresponding pressure signal P1, and the pressure signal P1 may be provided to the processor 12 after being processed by the low-pass filter 132 and the analog-to-digital converter 133. In addition, the inflation pressurization and deflation depressurization actions of the air pressure sensing circuit 13 may be carried out according to the instructions of the processor 12.

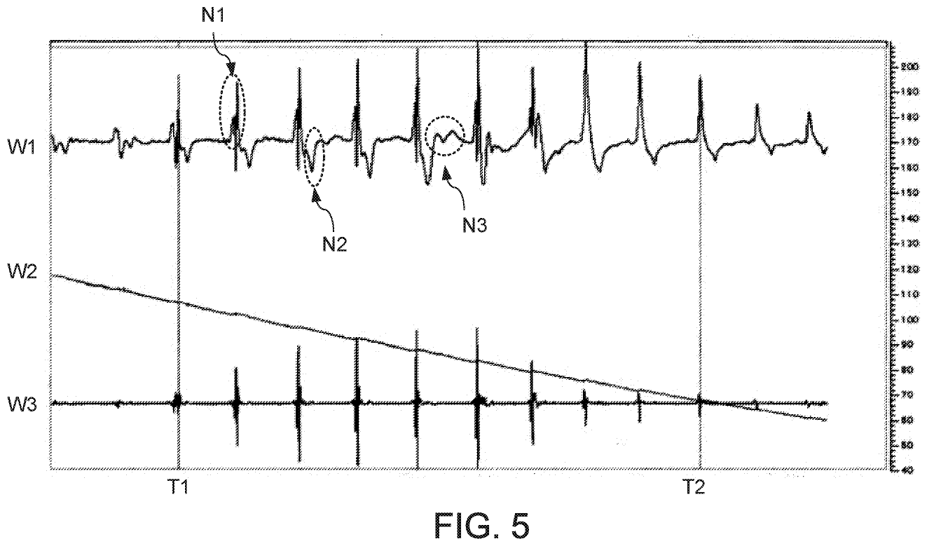

Next, referring to FIG. 5, the digital signal S1 before and after a filtering process by the processor 12 may be shown as a waveform W1 and a waveform W3 respectively, while the pressure signal P1 generated by the air pressure sensing circuit 13 may be shown as a waveform W2. In FIG. 5, the vertical axis represents the pressure in millimeters of mercury (mmHg), while the horizontal axis represents time; however, the pressure magnitude of the vertical axis shown in FIG. 5 is only applicable to the waveform W2, and the waveforms W1 and W3 are only superimposed around the waveform W2 so as to comprehensively determine the temporal correlations between the pressure change shown in the waveform W2 and the characteristics shown in the waveforms W1 and W3.

Much noise similar to the notch N2 and the notch N3 can be observed in the waveform W1, and the noise is very unfavorable to the subsequent blood pressure determination processes. For example, the notch N2 and the notch N3 are very likely to cause misjudgment when evaluating the appearance position of Korotkoff sound or determining the systolic pressure determination time point and the diastolic pressure determination time point via identifying the appearance and disappearance of the notch N1.

To solve the problem of misjudgment described above, the processor 12 may perform a filtering process on the digital signal S1 after receiving the digital signal S1. Specifically, the filtering process may comprise filtering out the noise around a principal component wave corresponding to the pulsation of the target area within a specific range in the digital signal S1. For example, the specific range may be a period of time after the start of depressurization of the pulse pressing element B1, for example, the range shown in FIG. 5 is about 15 seconds. In the practice of blood pressure measurement, a common subject will be pressurized to about 180 mmHg, while a subject with hypertension may be pressurized to about 200 to 240 mmHg, and the rate of depressurization may be about 4 to 5 mmHg per second, and the overall depressurization may be completed in about 20 to 30 seconds. In addition, the term “principal component wave” refers to the wave containing the features to be analyzed (e.g., the notch N1 in the waveform W1 and other features with the same structure.) The term “noise” refers to the signal content that may cause misjudgment, such as the notch N2 and the notch N3 in the waveform W1.

To filter out the noise, in some embodiments, the filtering process may comprise filtering out signal components with frequencies higher than 70 Hz in the digital signal S1. In some embodiments, the filtering process may also comprise further filtering out the signal components with frequencies lower than 15 Hz in the digital signal S1, that is, the filtering process retains the contents between 15 Hz and 70 Hz.

In some embodiments, the processor 12 may perform the filtering process by implementing a finite impulse response (FIR) digital filter, and the FIR digital filter may be set to a band-pass mode.

Through the above-mentioned filtering process, the processor 12 significantly removes the existence of the notch N2 and the notch N3, while at the same time, it retains the feature distribution that is consistent with the original features represented by the notch N1 in the waveform W1. As seen after repeated experiments of the inventor, the feature distribution in the waveform W3 (i.e., the distribution of major impulses) tends to be consistent with the feature distribution of the Korotkoff sound obtained through the artificial auscultation. Accordingly, the processor 12 may then determine a systolic pressure determination time point T1 and a diastolic pressure determination time point T2 according to this feature distribution form in the waveform W3, for example, determine the position where the first Korotkoff sound feature appears as the systolic pressure determination time point T1, and determine the position of the last identified Korotkoff sound feature as the diastolic pressure determination time point T2.

In some embodiments, the processor 12 may determine whether there is a Korotkoff sound feature in the waveform W3 by itself. Specifically, due to the fact that the waveform W3 in FIG. 5 is formed by the accumulation of numerical values of a group of discrete data, the processor 12 may determine whether each impulse in the waveform W3 belongs to a Korotkoff sound feature according to the comparison result between the numerical variation amount of this group of discrete data in a certain time interval and a threshold value. For example, the time interval may be 15 seconds, and the threshold value corresponding to the variation amount may be 10%. That is, the processor 12 may accordingly regard the impulse position corresponding to the time interval in the waveform W3 as having the Korotkoff sound feature when the numerical value of a group of discrete data corresponding to the waveform W3 changes by more than 10% within 15 seconds.

After determining the systolic pressure determination time point T1 and the diastolic pressure determination time point T2, the processor 12 may generate a blood pressure measurement result E1 according to the systolic pressure determination time point T1, the diastolic pressure determination time point T2, and the pressure signal P1 corresponding to the waveform W2. For example, in the example of FIG. 5, the blood pressure values measured at the systolic pressure determination time point T1 and the diastolic pressure determination time point T2 are 107 mmHg and 68 mmHg respectively.

In some embodiments, the processor 12 may also calculate the heart rate according to the peak interval in the waveform W3 and incorporate the heart rate information into the blood pressure measurement result E1.

In some embodiments, the device 1 for measuring blood pressure may further comprise an input/output (I/O) interface 14 electrically connected with the processor 12, and the I/O interface 14 may be a transmission interface such as USB, HDMI, Thunderbolt, Lightning, DisplayPort, 3.5/2.5 cm audio hole of all generations, without being limited thereto. The device 1 for measuring blood pressure may present the blood pressure measuring result E1 to the user through devices electrically connected to the input/output interface 14 (such as a display, a speaker, a vibrator (providing information to visually impaired and hearing-impaired people in the form of vibration) or the like).

Note that the electrical connection between the elements involved inside and/or outside the device 1 for measuring blood pressure described above may be direct (i.e., elements being connected to each other without other elements in between) or indirect (i.e., elements being connected to each other through other elements).

Also note that the low-pass filter 114 and the processor 12 perform different filtering processes respectively, and each of them has different uses and implementation methods. First, the low-pass filter 114 performs the filtering process on the analog signal from the differential signal amplifier 113, while the processor 12 performs the filtering process on the digital signal from the analog-to-digital converter. What are more important is that the purpose of the low-pass filter 114 is only to filter out the high-frequency components in the amplified signal from the differential signal amplifier 113, and that the low-pass filter 114 is only intended to generally retain the principal component wave corresponding to the pulsation of human body, and thus, as shown in the waveform W1 in FIG. 5, there is much noise around the principal component wave that are not favorable for the processor 12 to identify the characteristics of the principal component wave. Different from the low-pass filter 114, the digital filtering performed by the processor 12 is especially used for filtering out the noise around the principal component wave (e.g., the notch N2 and the notch N3 shown in FIG. 5), because these noises are not favorable for the processor 12 to identify the characteristics exhibited by the principal component wave; and as shown in FIG. 5, the waveform W3 specially filtered by the processor 12, as compared to the waveform W1, effectively reduces the noise around the principal component wave.

In some embodiments, the processor 12 may further use the generated waveform W3 as training data to train an artificial intelligence model and then identify the characteristics of Korotkoff sound in each input waveform with the trained artificial intelligence model.

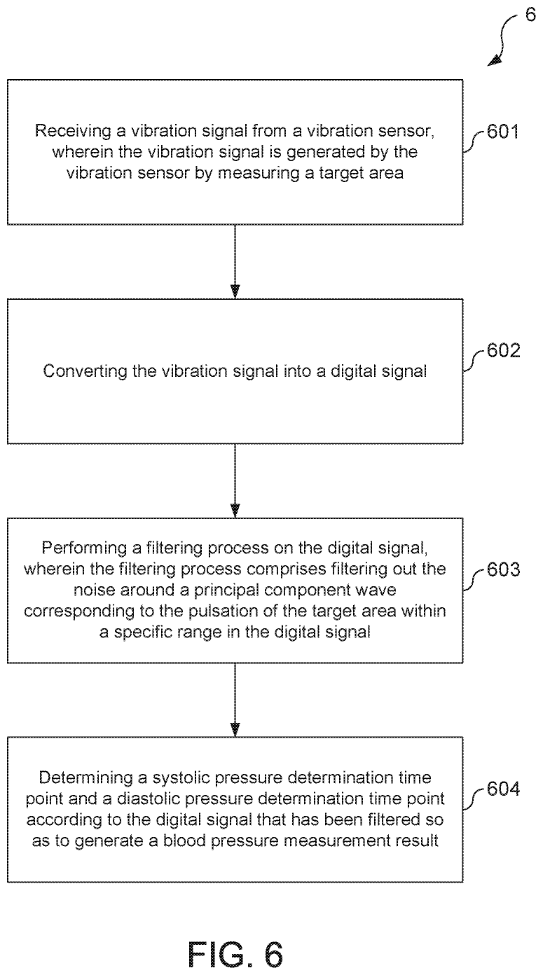

Referring to FIG. 6, a second embodiment of the present disclosure is a method 6 for measuring blood pressure, which may be executed by a device for measuring blood pressure and may comprise the following steps:
receiving a vibration signal from a vibration sensor, wherein the vibration signal is generated by the vibration sensor by measuring a target area (labeled as 601); and
converting the vibration signal into a digital signal (labeled as 602);
performing a filtering process on the digital signal, wherein the filtering process comprises filtering out the noise around a principal component wave corresponding to the pulsation of the target area within a specific range in the digital signal (labeled as 603); and
determining a systolic pressure determination time point and a diastolic pressure determination time point according to the digital signal that has been filtered so as to generate a blood pressure measurement result (labeled as 604).

In some embodiments, regarding the method 6 for measuring blood pressure, the filtering process may further comprise filtering out signal components with frequencies higher than 70 Hz in the digital signal. In some embodiments, the filtering process may further comprise filtering out signal components with frequencies below 15 Hz in the digital signal. In addition, in some embodiments, the device for measuring blood pressure may perform the filtering process by implementing a finite impulse response digital filter.

In some embodiments, the method 6 for measuring blood pressure may further comprise the following steps:
controlling a pulse pressing element to exert pressure on the target area; and
generating a pressure signal corresponding to the pulse pressing element, wherein the device for measuring blood pressure generates the blood pressure measurement result according to the systolic pressure determination time point, the diastolic pressure determination time point and the pressure signal.

In some embodiments, regarding the method 6 for measuring blood pressure, the vibration sensor may comprise a metallic diaphragm, and the metallic diaphragm may be made of copper alloy. Furthermore, in some embodiments, the copper alloy may be phosphor bronze. Also, in some embodiments, the vibration sensor may comprise a piezoresistive strain gauge.

Each embodiment of the method 6 for measuring blood pressure basically corresponds to a certain embodiment of the device 1 for measuring blood pressure. Therefore, all the corresponding embodiments of the method 6 for measuring blood pressure can be fully appreciated and realized by those of ordinary skill in the art simply with reference to the above description of the device 1 for measuring blood pressure, even though each embodiment of the method 6 for measuring blood pressure is not described in detail separately in the above description.

A third embodiment of the present disclosure is a computer program product implemented according to the method 6 for measuring blood pressure in the second embodiment. When the computer program product is read into an electronic computing device, the electronic computing device will execute the corresponding steps of each embodiment of the method 6 for measuring blood pressure described in the second embodiment. The computer program product refers to an article that carries a computer-readable program and is not limited in external forms, such as but not limited to a non-transitory tangible machine-readable medium, for example but not limited to a read-only memory (ROM), a flash memory, a floppy disk, a mobile hard disk, a magnetic tape, a network database, a cloud node, or any other computer software storage medium with the same function and well known to those of ordinary skill in the art.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

1. A device for measuring blood pressure, comprising:
a signal conversion circuit, being configured to:
receive a vibration signal from a vibration sensor, wherein the vibration signal is generated by the vibration sensor by measuring a target area; and
convert the vibration signal into a digital signal; and
a processor, being electrically connected with the signal conversion circuit, and being configured to:
perform a filtering process on the digital signal, wherein the filtering process comprises filtering out noise around a principal component wave corresponding to pulsation of the target area within a specific range in the digital signal; and
determine a systolic pressure determination time point and a diastolic pressure determination time point according to the digital signal that has been filtered, thereby generating a blood pressure measurement result.

2. The device for measuring blood pressure according to claim 1, wherein the filtering process further comprises filtering out signal components with frequencies higher than 70 Hz in the digital signal.

3. The device for measuring blood pressure according to claim 2, wherein the filtering process further comprises filtering out signal components with frequencies lower than 15 Hz in the digital signal.

4. The device for measuring blood pressure according to claim 1, wherein the signal conversion circuit comprises:
three resistors, constituting a Wheatstone bridge with the vibration sensor, the Wheatstone bridge being configured to convert the vibration signal into a pair of differential signals;
a differential signal amplifier, being electrically connected with the Wheatstone bridge for receiving the pair of differential signals from the Wheatstone bridge and converting the pair of differential signals into an amplified signal;
a low-pass filter, being electrically connected with the differential signal amplifier for filtering the amplified signal; and
an analog-to-digital converter, being electrically connected with the low-pass filter and the processor and being configured to convert the amplified signal that has been filtered into the digital signal.

5. The device for measuring blood pressure according to claim 1, further comprising:
a pulse pressing element, wherein the processor is further configured to control the pulse pressing element to exert pressure on the target area; and
an air pressure sensing circuit, being electrically connected with the processor and the pulse pressing element and being configured to generate a pressure signal corresponding to the pulse pressing element and provide the pressure signal to the processor, wherein the processor generates the blood pressure measurement result according to the systolic pressure determination time point, the diastolic pressure determination time point, and the pressure signal.

6. The device for measuring blood pressure according to claim 1, wherein the processor performs the filtering process by implementing a finite impulse response digital filter.

7. The device for measuring blood pressure according to claim 1, wherein the vibration sensor comprises a metallic diaphragm, and the metal diaphragm is made of copper alloy.

8. The device for measuring blood pressure according to claim 7, wherein the copper alloy is phosphor bronze.

9. The device for measuring blood pressure according to claim 7, wherein the vibration sensor comprises a piezoresistive strain gauge.

10. A method for measuring blood pressure, being executed by a device for measuring blood pressure, and comprising:
receiving a vibration signal from a vibration sensor, wherein the vibration signal is generated by the vibration sensor by measuring a target area;
converting the vibration signal into a digital signal;
performing a filtering process on the digital signal, wherein the filtering process comprises filtering out noise around a principal component wave corresponding to pulsation of the target area within a specific range in the digital signal; and
determining a systolic pressure determination time point and a diastolic pressure determination time point according to the digital signal that has been filtered so as to generate a blood pressure measurement result.

11. The method for measuring blood pressure according to claim 10, wherein the filtering process further comprises filtering out signal components with frequencies higher than 70 Hz in the digital signal.

12. The method for measuring blood pressure according to claim 11, wherein the filtering process further comprises filtering out signal components with frequencies lower than 15 Hz in the digital signal.

13. The method for measuring blood pressure according to claim 10, further comprising:
controlling a pulse pressing element to exert pressure on the target area; and
generating a pressure signal corresponding to the pulse pressing element, wherein the device for measuring blood pressure generates the blood pressure measurement result according to the systolic pressure determination time point, the diastolic pressure determination time point and the pressure signal.

14. The method for measuring blood pressure according to claim 10, wherein the device for measuring blood pressure performs the filtering process by implementing a finite impulse response digital filter.

15. The method for measuring blood pressure according to claim 10, wherein the vibration sensor comprises a metallic diaphragm, and the metallic diaphragm is made of copper alloy.

16. The method for measuring blood pressure according to claim 15, wherein the copper alloy is phosphor bronze.

17. The method for measuring blood pressure according to claim 15, wherein the vibration sensor comprises a piezoresistive strain gauge.

18. A computer program product, after being loaded into an electronic computing device, causing the electronic computing device to execute the following instructions:
receiving a vibration signal from a vibration sensor, wherein the vibration signal is generated by the vibration sensor by measuring a target area; and
converting the vibration signal into a digital signal;
performing a filtering process on the digital signal, wherein the filtering process comprises filtering out noise around a principal component wave corresponding to pulsation of the target area within a specific range in the digital signal; and
determining a systolic pressure determination time point and a diastolic pressure determination time point according to the digital signal that has been filtered so as to generate a blood pressure measurement result.

19. The computer program product according to claim 18, wherein the filtering process further comprises filtering out signal components with frequencies higher than 70 Hz in the digital signal.

20. The computer program product according to claim 19, wherein the filtering process further comprises filtering out signal components with frequencies lower than 15 Hz in the digital signal.

21. The computer program product according to claim 18, after being loaded into the electronic computing device, further causing the electronic computing device to execute the following instructions:
controlling a pulse pressing element to exert pressure on the target area; and
generating a pressure signal corresponding to the pulse pressing element, wherein the electronic computing device generates the blood pressure measurement result according to the systolic pressure determination time point, the diastolic pressure determination time point and the pressure signal.

22. The computer program product according to claim 18, wherein the electronic computing device performs the filtering process by implementing a finite impulse response digital filter.
